(19) United States
(12) Patent Application Publication
(10) Pub. No.: US 2004/0052853 A1
(43) Pub. Date: Mar. 18, 2004

(54) Pectin films

(75) Inventor: Ross Clark, San Diego, CA (US)

Correspondence Address: BANNER & WITCOFF, 1001 G STREET N W, SUITE 1100, WASHINGTON, DC, 20001, US

(73) Assignee: CP Kelco, U.S., Inc., Wilmington, DE (US)

(21) Appl. No.: 10/635,439

(22) Filed: Aug. 7, 2003

Related U.S. Application Data
(60) Provisional application No. 60/410,811, filed on Sep. 16, 2002.

(51) Int. Cl. A61K 31/736; A61K 9/14

(52) U.S. Cl. 424/488; 514/54

(57) ABSTRACT

Pectin films are treated to alter their dissolution characteristics. More specifically, films can be made to dissolve more quickly by reducing the molecular weight of the starting pectin. Applications of the pectin films include drug delivery and breath films.

### CROSS REFERENCE TO RELATED APPLICATION

This application claims priority of U.S. Provisional Application Serial No. 60/410,811 entitled “PECTIN FILMS” filed Sep. 16, 2002, which is incorporated herein by reference.

### FIELD OF THE INVENTION

The invention relates to low molecular weight pectin films having rapid dissolution rates.

### BACKGROUND OF THE INVENTION

Films formed from water-soluble polymers can be used to administer various pharmaceutical compounds, including drugs and orally active agents such as breath fresheners. Examples of such polymers are disclosed in, for example, U.S. Pat. Nos. 6,419,903; 6,284,264; 6,177,096; and 5,948,430.

Polymer solutions are cast onto a suitable surface and then dried to form a film. More specifically, the film is formed by mixing a high concentration polymer solution or “dope” and then adding the active agents along with plasticizing agents and other ingredients. A thin film or shape is cast and the final product results from drying this film or shape.

The use of pectin and many other water-soluble polymers are well known, see for example U.S. Pat. No. 6,197,331. A pectin solution can be formed from many types of pectin including high methoxyl, low methoxyl, and low methoxyl-amidated type pectins. Usually the pectin solution is deposited at a high concentration of pectin (from 3 wt % to as much as 15 wt % pectin solids). The pectin is cast on a suitable metal or polymeric surface to deposit a smooth film, without voids, which can be removed from the surface with minimal damage. Drying can be carried out by any suitable technique such as room temperature air-drying, heated belt drying, and refractive window drying on a Mylar or similar plastic belt.

However, pectin doesn't easily dissolve in the mouth or aqueous solutions. Thus, pectin is not generally used for the quick release of a pharmaceutical or orally active agents in the oral cavity.

### BRIEF SUMMARY OF THE INVENTION

It was discovered that the dissolution rate of pectin can be modified so that the pectin will dissolve in aqueous media and in the mouth more rapidly. In particular, the pectin solutions can be treated to reduce the molecular weight in such a way to render the pectin solution suitable for film casting and quicker dissolution. Modifying the molecular weight of the pectin allows an increase in pectin solution solids and aids in dissolution of the film once the film has been formed. This in turn allows an increased dissolution rate and also provides the pectin with better flavor properties and a less gummy mouthfeel.

Thus the invention is directed to an orally consumable film composition for delivering at least one active agent to an oral cavity wherein the film is rapidly dissolvable in the oral cavity, the composition comprising a high or low methyl ester pectin having an intrinsic viscosity of about 2.5 dl/g or less, preferably about 1.8 dl/g or less, and at least one active agent.

The rapidly dissolvable pectin films are useful for, for example, drug delivery and breath films. The pectin films formed in accordance with the instant invention have an adjustable dissolution rate, have good hydration to make casting “dope,” and do not have off odors or flavors. In addition, the films are consistently strong and flexible, do not mask flavors or perfumes, and are compatible with active agents. The application of the quick-release composition is easy and uncomplicated.

The invention is directed to a rapidly dissolving pectin film wherein the pectin is a high methyl ester (“HM”) pectin having a low molecular weight as evidenced by having an intrinsic viscosity of about 2.5 dl/g or less, preferably about 1.8 dl/g or less. Any suitable HM pectin may be used.

The invention is further directed to a rapidly dissolving pectin film wherein the pectin is a low methyl ester (“LM”) pectin having a low molecular weight as evidenced by having an intrinsic viscosity of about 2.5 dl/g or less, preferably about 1.8 dl/g or less. Any suitable LM pectin may be used. Preferably the LM pectin is an amidated LM (“LMA”) pectin.

The invention is further directed to a process for making a rapidly dissolving pectin film, comprising the steps of: obtaining a pectin having a molecular weight as evidenced by having an intrinsic viscosity of about 4.9 dl/g. or more; degrading the pectin's molecular weight in order reduce the pectin's molecular weight as evidenced by having an intrinsic viscosity of about 2.5 dl/g or less, preferably about 1.8 dl/g. or less, to form a low molecular weight pectin; and forming a film from the low molecular weight pectin.

### BRIEF DESCRIPTION OF THE DRAWINGS

FIG. 1 depicts one embodiment for preparing the pectin films.

FIG. 2 depicts one embodiment for reducing the intrinsic viscosity of a pectin solution.

### DETAILED DESCRIPTION OF THE INVENTION

The present invention is directed to a film that rapidly dissolves orally in the mouth and releases a pharmaceutically or orally active agent, such as a flavorant. The film comprises pectin having reduced molecular weight and one or more pharmaceutically or orally active ingredients.

It was discovered that when the molecular weight of pectin is reduced, a film prepared with the pectin dissolves in aqueous media and in the mouth more rapidly. Advantageously, this reduction in molecular weight also allows the initial pectin solution to be prepared at a higher solids loading while keeping the viscosity within manageable limits. A higher concentration of pectin means that less water has to be removed during film formation.

Also advantageously, reducing the molecular weight of the pectin also allows films to be formed, which are more flexible or plastic that those made from higher molecular weight polymers. Smaller and more flexible polymer chains generally allow films which are more flexible and do not break when bent into a “U” or hairpin shape.

This reduces the need for additional plasticizer, such as polyethylene glycol (PEG) and glycerin, which can make films tacky and stick together under high humidity condition. Although small amounts in plasticizing agents can enhance the extensibility of the film and thus increase its strength, larger amounts will soften the film excessively.

Generally, polymers having a higher viscosity will produce stronger films. Those with a more regular structure will also form films with high tensile strength. Excess strength of what is required is not desired as such strong films tend to dissolve slowly and release active materials at a lower rate. If the molecular weight of a film is too low, the film may become brittle such as a film made with sucrose.

Critical to the function of any film is achieving the proper rate of film dissolution. The inventive process modifies the viscosity of the pectin to allow the film to dissolve very quickly and completely. A film that dissolves more quickly will release the active agent contained in the film more rapidly. This is desired, for example, for a flavor with an initial high impact.

Films prepared from pectin solutions treated to reduce their molecular weight as evidenced by intrinsic viscosity dissolve much more quickly in the mouth and release the flavor much more readily than do films prepared from untreated pectin. Rapid dissolution and release of flavor are especially important in applications such as breath films. Slower rates of dissolution can be obtained by degrading the pectin less extensively or by addition of another high molecular weight polymer. When as little as 0.1% xanthan gum is added to the pectin, the dissolution rate is slowed to a noticeable degree.

Films made with reduced molecular weight pectin have a greater intensity of flavor than prior art films and the flavor remains at high levels during storage. Moreover, pectin is available in USP grades and has few regulatory barriers around the world. Some competitive products such as gelatin, modified starch and synthetic polymers have more regulatory barriers.

The pectin film may be formed from many types of pectin including high methoxyl, low methoxyl, and low methyl ester amidated type pectins. Preferably, high methoxyl pectins are used. The galacturonic acid residues in pectin are partly esterified and present as the methyl ester. The degree of esterification is defined as the percentage of carboxyl groups esterified. Pectin with a degree of esterification (“DE”) above 50% is named high methyl ester (“HM”) pectin or high ester pectin and one with a DE lower than 50% is named low methyl ester (“LM”) pectin or low ester pectin. Most pectin found in fruits and vegetables are HM pectins.

A variety of techniques can be used to reduce molecular weight in pectin solutions. Some of these include mechanical means such as homogenization at high pressures, neutralization of the pectin pH with a suitable base or caustic, for example sodium hydroxide, or with various oxidizing agents such as peroxide, e.g. 5% hydrogen peroxide. Enzymes designed to cleave the polymer backbone may also be used such as pectinase. The choice of the oxidizing agent to reduce the molecular weight will be dictated by the process in question, the costs of the ingredients, and other factors, in order to obtain the desired pectin with a reduced molecular weight and improved solubility.

Neutralization of pectin with an alkaline material such as sodium hydroxide is an effective way of reducing the molecular weight of pectin. A process referred to as “beta elimination” occurs when the pH of a pectin solution is raised from its normal range of 3-4 to 6-8. This reaction results in cleavage of the glycosidic linkages that polymerize the pectin. After 5 minutes or more at a temperature of 35° C. or more, the molecular weight of the pectin is measurably reduced by this process. This reaction proceeds more rapidly with the HM (high methoxyl) grades of pectin. The preferred conditions for reducing the molecular weight of the pectin are 50 to 65° C. at a pH value of 6.5 to 7.5 with a starting pectin solution concentration of 5 to 10% by weight. The process can be carried on for some time but the most preferred times at 15 to 30 minutes. Adjustment of the pH from the starting value of 3-4 is achieved with a 10% NaOH solution. Other alkaline materials can also be used. The pH change is what triggers the reaction, not the specific material used to change the pH. The resulting molecular weight reduction results in films with enhanced solubility as compared to the starting pectin.

HM pectin may be degraded by homogenizing at a pressure of 5,000 psi for 5 passes through a homogenizer. Alternatively, HM pectin may be degraded through the use of an oxidizer, preferably 5% hydrogen peroxide. HM pectin may also be degraded using a depolymerizing enzyme such as the pectinase made from Aspergillus niger available from Worthington Biochemical Corporation 730 Vassar Ave Lakewood, N.J., 08701. LM pectin or LMA pectin also may be degraded using mechanical, chemical or enzymatic means.

The degree of degradation in the molecular weight of the pectin may be determined by the method of intrinsic viscosity measurement. Pectin that has been sufficiently degraded will have an intrinsic viscosity of less than 2.5 dl/g, preferably less than 1.8 dl/g, when measured in a 0.05 M pH 3.75 citrate buffer system.

One suitable HM pectin is D Slow Set (available from CP Kelco ApS) that has been degraded by neutralization to pH 6.5 in the use of rapidly dissolving films. The starting intrinsic viscosity is about 4.9 dl/g and the final product has an intrinsic viscosity of 1.8 dl/g. A standard pullulan used in this application has an intrinsic viscosity of 0.9 dl/g.

The resulting film has instant wettability, which allows the film to soften immediately after application to the mucosal tissue thus preventing the patient from experiencing any prolonged adverse feeling in the mouth. The film also has a tensile strength suitable for normal coating, cutting, slitting, and packaging operations.

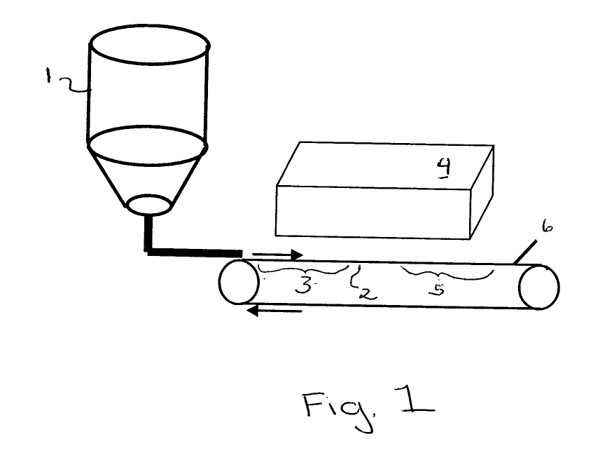

FIG. 1 shows one embodiment of the film making operation. First the polymer solution or dope is prepared and mixed well to obtain a high polymer loading. The mixing tank (1) may be temperature controlled and a second surge tank may be plumbed into the system to allow for continuous operation.

Following the mixing step, the solution is cast (evenly distributed) on a releasable carrier such as a belt (2). A manifold, blades, nozzles, and/or other suitable equipment may be used to cast the polymer. The carrier material must have a surface tension, which allows the film solution to spread evenly across the intended carrier width without soaking to form a destructive bond between the film and carrier substrates. Examples of suitable carrier materials include glass, stainless steel, Teflon and polyethylene-impregnated paper.

It is desirable to have the casting solution or dope at as high a polymer loading as possible. This leaves less water to be removed in the drying process and leads to a film that will curl less and dry more quickly. Quick drying will also preserve volatile ingredients like flavors. Pectin may be prepared to concentrations of 10% or even more when properly treated. Depending upon the system used to cast the film onto the belt, the ideal viscosity of the dope can range from 5,000 cp to over 50,000 cp. The dope may also be heated before putting in on the belt, which reduces viscosity, decreases drying times and eases material handling.

The cast film should be of even thickness and quite a bit thicker than the final film. As it dries, the film will not contract so much from side to side as it will become thinner in cross section. For example, a film with a final thickness of 0.0015 inches (1.5 mils) and a solids loading of the dope of 10%, should have an initial film thickness of about 0.012 (12 mils).

The composition is cast on the releasable carrier and dried. Drying of the film may be carried out at high temperature using a drying oven, drying terminal, vacuum drier, or any other suitable drying equipment, which does not adversely affect the ingredients of which the film is composed.

In the figure, the cast film on the belt passes over a heating section (3). Heat can be applied in any suitable manner such as steam or infrared radiation. The cast film and belt can be heated from below or above or even both sides. Typically, a hood (4) or other arrangement is used over the belt to remove the moisture and control the environment while drying. Preferably, the drying will be monitored such as with a reflected infrared instrument. Excessive heat should be avoided since it causes the film to curl after storage. Water activity of the product when dried should be about 0.3 to 0.5 (30% to 50% RH). The cast film on the belt may then pass over a cooling section (5). Cooling may be used after the heating step to prepare the film for removal from the belt.

The film is then removed from the carrier in any suitable manner. In the figure, the film is removed from the belt with a scraper blade (6). Clean removal from the belt is essential for a smooth running operation. To aid in removal, a surfactant may be applied to the belt before drying starts. In addition, because the pectin has a low viscosity and high polymer loading, removal is relatively easy. Excessive amounts of plasticizing agent should be avoided as it will cause the film to stick to the belt.

Once removed, the film may be cut into strips and wound onto rolls. The film may also be segmented into dosage units by die-cutting or slitting-and-die-cutting. The segmented film has a strip width of generally about 15 to about 30 millimeter and a length of about 20 to about 50 millimeters in length. The film has a thickness ranging from about 10 to about 200 micrometers, and preferably about 25 to 75 micrometers.

The film is preferably shaped and sized for placement in the mouth. The film is flexible and adheres to a surface in the mouth, usually the roof of the mouth or the tongue, and quickly dissolves, typically in less than two minutes, preferably from 15 seconds to one minute. Dissolution speed can be controlled thickness of the film.

The pectin film in accordance with the invention has a dissolution rate of at least 10%, preferably at least 25%, more preferably at least 35%, over untreated pectin. Typically improvements range from 25 to 50% improvement.

The low molecular weight pectin and other water-soluble polymers can be used to administer various pharmaceutical compounds, such as drugs and breath fresheners.

The film can further comprise water, additional film forming agents, plasticizing agents, flavoring agents, antimalodor agents, surfactants, emulsifying agents, coloring agents, sweeteners and fragrances.

Flavoring agents include those known to the skilled artisan, such as natural and artificial flavors. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. These flavor agents can be used individually or in admixture. Commonly used flavors include mints such as peppermint, artificial vanilla, cinnamon derivatives, and various fruit flavors, whether employed individually or in admixture. Generally, any flavoring or food additive, such as those described in Chemicals Used in Food Processing, publication 1274 by the National Academy of Sciences, pages 63-258, may be used. The amount of flavoring agent employed is normally a matter of preference subject to such factors as flavor type, individual flavor, and strength desired. Generally the flavoring is incorporated in the film of the present invention in an amount ranging from about 2.0 to about 20% by weight and preferably about 5 to about 10% by weight.

Sweeteners useful in the practice of the present invention include both natural and artificial sweeteners. Suitable sweetener include water soluble sweetening agents such as monosaccharides, disaccharides and polysaccharides such as xylose, ribose, glucose (dextrose), mannose, glatose, fructose (levulose), sucrose (sugar), maltose, water soluble artificial sweeteners such as the soluble saccharin salts, i.e., sodium or calcium saccharin salts, cyclamate salts dipeptide based sweeteners, such a L-aspartic acid derived sweeteners, such as L-aspartyl-L-phenylalaine methyl ester (aspartame) or other sweeteners such as Sucralose.

The compositions of the present invention can also contain coloring agents or colorants. The coloring agents are used in amounts effective to produce the desired color and include natural food colors and dyes suitable for food, drug and cosmetic applications. These colorants are known as FD&C dyes and lakes. The materials acceptable for the foregoing spectrum of use are preferably water-soluble, and include FD&C Blue No.2, which is the disodium salt of 5,5-indigotindisulfonic acid. Similarly, the dye known as Green No.3 comprises a 15 triphenylmethane dye and is the monosodium salt of 4-[4-N-ethyl-p-sulfobenzylamino) diphenyl-methylene ]-[1-N-ethy 1-N-sulfonium benzyl)-2,5-cyclo-hexadienimine]. A full recitation of all FD&C and D&C dyes and their corresponding chemical structures may be found in the Kirk-Othrner Encyclopedia of Chemical Technology, Volume 5, Pages 857-884, which text is accordingly incorporated herein by reference.

Active breath freshening agents can be incorporated into the film composition of the present invention to form the breath freshening strips of the present invention. The active ingredients include zinc gluconate, zinc citrate and/or alpha ionone. These agents function in masking mouth odor and reducing volatile odor causing bacterial sulfur compounds. These agents may be incorporated in the film composition of the present invention at a concentration of about 0.1 to about 2.0% by weight and preferably about 0.15 to about 0.5% by weight.

In general, the effective amount of sweetener is utilized to provide the level of sweetness desired for a particular composition, will vary with the sweetener selected. This amount will normally be about 0.01% to about 2% by weight of the composition.

### EXAMPLE 1

A solution of CP Kelco D Slow Set pectin of 14% concentration was prepared in deionized water by mixing at 65° C. Once fully dissolved, the pectin solution was neutralized with 10% NaOH to a pH value of 6.5. This had the effect of greatly thinning the solution. Plasticizer agents (glycerin 1% and PEG-200 0.5%) were added along with 2.3% flavor oil and 0.4% sodium saccharin. This solution was cast on plastic (rigid polystyrene) surface using a doctor blade device. The initial film was 0.018 inches thick and it dried to 0.0015 inches thick after 24 hours at room temperature and humidity.

### EXAMPLE 2

The solutions in this example were neutralized from the standard acidic pH to one near neutrality. This caused a decrease in the molecular size of the pectin, allowing a solutions with a higher solids levels to be made, and advantage when preparing starting materials for a dope to be cast into a film. Films made from the modified pectin also dissolved more quickly when exposed to water. This gives a better mouthfeel and causes the flavor to be perceived more quickly.

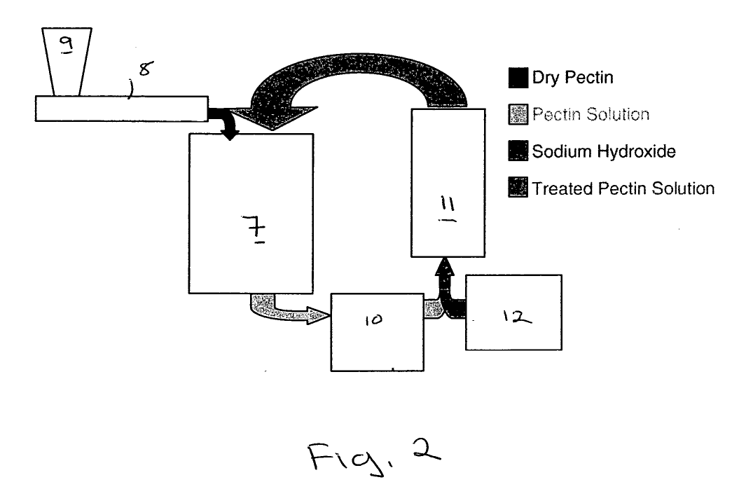

As shown in FIG. 2, the solution is prepared in a mixing tank (7). The pectin powder is added to the open top of the tank using a vibratory feeder (8) and funnel (9). The tank is equipped with a variable frequency drive to adjust the mixing speed, and a pump (10) is used to circulate the pectin solution. The pump is preferably a large positive displacement driven by a variable frequency drive motor to control speed.

A colloid mill (11) is used with an injection T added to the inlet of the mill. The caustic is added in a mixing T using a positive displacement pump (12) with a variable frequency drive.

| | Kg | % |
|---|---|---|
| Water* | 75.8 | 84.3% |
| Pectin | 11 | 12.2% |
| Na Citrate | 0.087 | 0.1% |
| Methyl paraben | 0.087 | 0.1% |
| 12.5% NaOH** | 3 | 3.3% |
| Total | 89.974 | 100.0% |

*Measured as 20 gallons
**Quantity of NaOH is estimated, amount used is dictated by pH measurement
Does not account for evaporative losses that are expected due to 50-60 minute run temperatures.

Twenty gallons of tap water are added to the mixing tank at a temperature of about 55° C.+/−3° C. The speed of the mixing tank is set to about 25 Hz. Eleven kilograms of pectin are gradually added to the mixing tank using the vibratory feeder. The pectin is added to the vortex in the mixer. Water is passed through the colloid mill and sodium citrate is added to the water. The mix speed is gradually increased as the pectin is added. The mixer should reach about 60 Hz in about 7 or 8 minutes.

After about 12 minutes, typically about 8-9 kg of the total 11 kg of pectin is in solution but the mixture is no longer circulating well due to the high viscosity. A small amount of caustic is added to lower the viscosity by turning off the pectin feed, turning on the NaOH positive displacement and setting the speed to 40-45%. In about 2 minutes, the viscosity drops enough to add the pectin again. The NaOH positive displacement remains on and set to 40-45%.

Pectin and NaOH are added such that the NaOH dropped the viscosity at about the same rate that the dissolving pectin is increased (about 14 minutes). Ideally, the viscosity is held at nearly the maximum that the mixer can mix. NaOH should not be added at rate high enough to thin the product excessively at this time.

After about 22 minutes, all the pectin should be in, the temperature should be about 58-60° C. and the viscosity should be very high, near the maximum the mixer can mix. The remaining caustic is added to adjust the pH to the final point.

The product positive displacement is set to 20 Hz to increase the flow rate through the colloid mill. The pressure should not exceed 20 psi. The feed rate on the caustic positive displacement is increased to about 55-60%. The product should not appear at all yellow or green, which would indicate the rate of caustic addition is too high.

The pH of the material in the tank and from the exit of the colloid mill is monitored, preferably with a flat surface pH electrode. The pH should initially be less than 5 and will rise slowly for most of the caustic addition. The material exiting from the mill should always remain at a pH lower than 7.5. During this period, the speed of the mixer will need to be reduced until, near the end of the process, the mixer speed is about 15 Hz. The temperature should be about 60-63° C.

As the process nears completion, the pH will begin to increase rapidly. After about thirty minutes, the tank pH should reach about 6.5, and the caustic pump should be stopped. Methylparaben is added and mixed for 2-3 minutes to insure uniformity. The product is screened using a 60 mesh screen and is stored in containers while hot.

The feeders may be any suitable feeder such as a screw feeder or a vibratory feeder. The positive displacement pumps may be any suitable pump such as peristaltic pumps or progressive cavity pumps.

The caustic should not be in contact with the pectin except under the high shear of the mill. A high flow rate is desired through the mill so that the caustic addition is gradual and no single unit volume of the pectin is over-treated with base. The flow rate should be set so that the mixer “turns over” in about 2 minutes or so. Preferably the end point is determined by viscosity, not pH. Citric acid may be added at the end of the process to drop the pH to about 5 and assure that no further degradation occurs.

Suitable pectin films within the invention may be obtained by substituting the generically and specifically described constituents and/or operating conditions of this invention for those used in the preceding examples.

While the invention has been described with respect to specific examples including presently preferred modes of carrying out the invention, those skilled in the art will appreciate that there are numerous variations and permutations of the above described systems and techniques that fall within the spirit and scope of the invention.

I claim:

1. An orally consumable film for delivering at least one active agent to an oral cavity wherein the film is rapidly dissolvable in the oral cavity, wherein the film is produced from a composition comprising a pectin having an intrinsic viscosity of about 2.5 dl/g or less and at least one active agent.

2. The film of claim 1 wherein the intrinsic viscosity is about 1.8 dl/g or less.

3. The film of claim 1 wherein the pectin is a high methyl ester pectin.

4. The film of claim 1 wherein the pectin is a low methyl ester pectin.

5. The film of claim 1 wherein the pectin is present at a concentration of about 20 to about 80% by weight based on the dry weight of the film.

6. The film of claim 1 wherein the active agent is a flavoring agent.

7. The film of claim 1 wherein the active agent is a pharmaceutical.

8. A composition for producing an orally consumable film for delivering at least one active agent to an oral cavity which is rapidly dissolvable in the oral cavity, the composition comprising a pectin having an intrinsic viscosity of about 2.5 dl/g or less and at least one active agent.

9. The composition of claim 8 wherein the intrinsic viscosity is about 1.8 dl/g or less.

10. The composition of claim 8 wherein the pectin is a high methyl ester pectin.

11. The composition of claim 8 wherein the pectin is a low methyl ester pectin.

12. The composition of claim 8 wherein the active agent is a flavoring agent.

13. The composition of claim 8 wherein the active agent is a pharmaceutical.

14. A method for delivering a breath freshening agent to an oral cavity which comprises preparing an orally consumable film composition which is rapidly dissolvable in the oral cavity, the composition comprising a pectin having an intrinsic viscosity of about 1.8 dl/g or less and at least one active agent, and placing the film in the oral cavity.

15. The method of claim 14 wherein the intrinsic viscosity is about 1.8 dl/g or less.

16. A process for making a rapidly dissolving pectin film, comprising the steps of: degrading a first pectin solution having an intrinsic viscosity of about 4.9 dl/g or more to obtain a second pectin solution having an intrinsic viscosity of about 2.5 dl/g or less; and forming a film from the second pectin solution.

17. The process of claim 16 wherein the intrinsic viscosity is about 1.8 dl/g or less.

18. The process of claim 16 further comprising adding an active ingredient to the first or second pectin solution.

19. The process of claim 16 wherein the first pectin solution is degraded by homogenizing.

20. The process of claim 16 wherein the homogenizing is at a pressure of about 5,000 psi.

21. The process of claim 16 wherein the first pectin solution is degraded by an oxidizer.

22. The process of claim 21 wherein the oxidizer is 5% hydrogen peroxide.

23. The process of claim 16 wherein the first pectin solution is degraded by depolymerizing enzyme.

24. The process of claim 23 wherein the enzyme is pectinase.

25. The process of claim 16 wherein the first pectin solution is degraded by raising the pH.

26. The process of claim 25 wherein the pH is raising with sodium hydroxide.

27. The process of claim 25 wherein the pH is raised to about 6 to about 8.
